(19) United States
(12) Patent Application Publication
(10) Pub. No.: US 2016/0302962 A1
(43) Pub. Date: Oct. 20, 2016

(54) Contraception Container Assembly

(71) Applicants: Jessica Rivera, Lakewood, NY (US); Enrique Bermudez, Lakewood, NY (US)

(72) Inventors: Jessica Rivera, Lakewood, NY (US); Enrique Bermudez, Lakewood, NY (US)

(21) Appl. No.: 14/687,320

(22) Filed: Apr. 15, 2015

(51) Int. Cl. A61F 6/00; B65D 55/02; B65D 51/24; B05B 11/00; B65D 81/22

(52) U.S. Cl. CPC A61F 6/005; B05B 11/3042; B65D 81/22; B65D 51/24; B65D 55/02

(57) ABSTRACT

A contraception container assembly for includes a housing that may insertably receive a contraceptive device. A reservoir is positioned within the housing and the reservoir may contain a fluid. A spraying unit is attached to the housing and the spraying unit is in fluid communication with the reservoir. Thus, the spraying unit urges the fluid from the reservoir into the housing thereby facilitating the contraceptive device to be immersed in the fluid.

### BACKGROUND OF THE DISCLOSURE

1. Field of the Disclosure

The disclosure relates to container devices and more particularly pertains to a new container device for storing a contraceptive device having the contraceptive device immersed in a fluid.

2. Summary of the Disclosure

An embodiment of the disclosure meets the needs presented above by generally comprising a housing that may insertably receive a contraceptive device. A reservoir is positioned within the housing and the reservoir may contain a fluid. A spraying unit is attached to the housing and the spraying unit is in fluid communication with the reservoir. Thus, the spraying unit urges the fluid from the reservoir into the housing thereby facilitating the contraceptive device to be immersed in the fluid.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a top perspective view of a contraception container assembly in an open according to an embodiment of the disclosure.

FIG. 2 is a perspective view of an embodiment of the disclosure in a closed position.

FIG. 3 is a bottom phantom view of an embodiment of the disclosure.

FIG. 4 is a front phantom view of an embodiment of the disclosure.

FIG. 5 is a back view of an embodiment of the disclosure.

FIG. 6 is a schematic view of an embodiment of the disclosure.

### DESCRIPTION OF THE PREFERRED EMBODIMENT

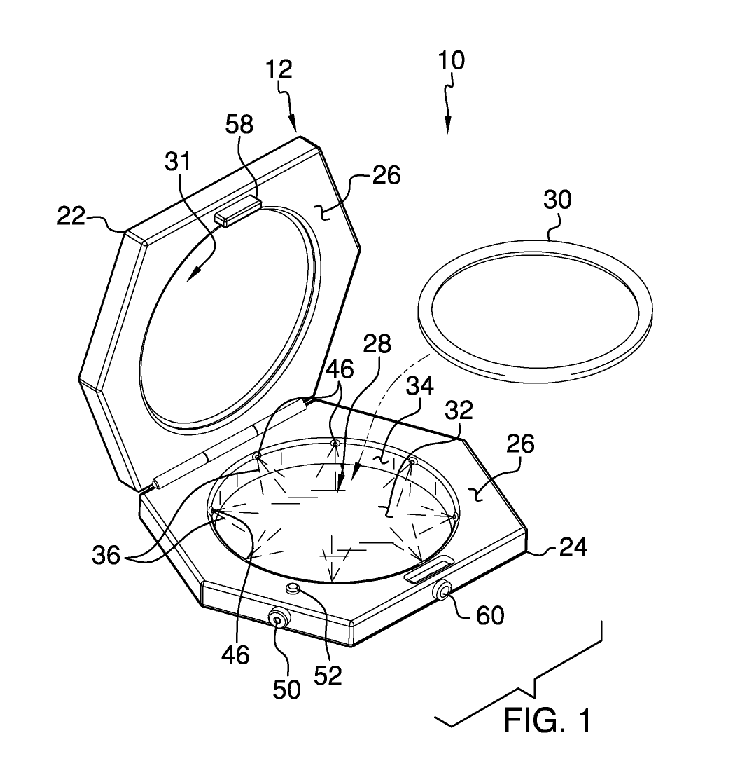

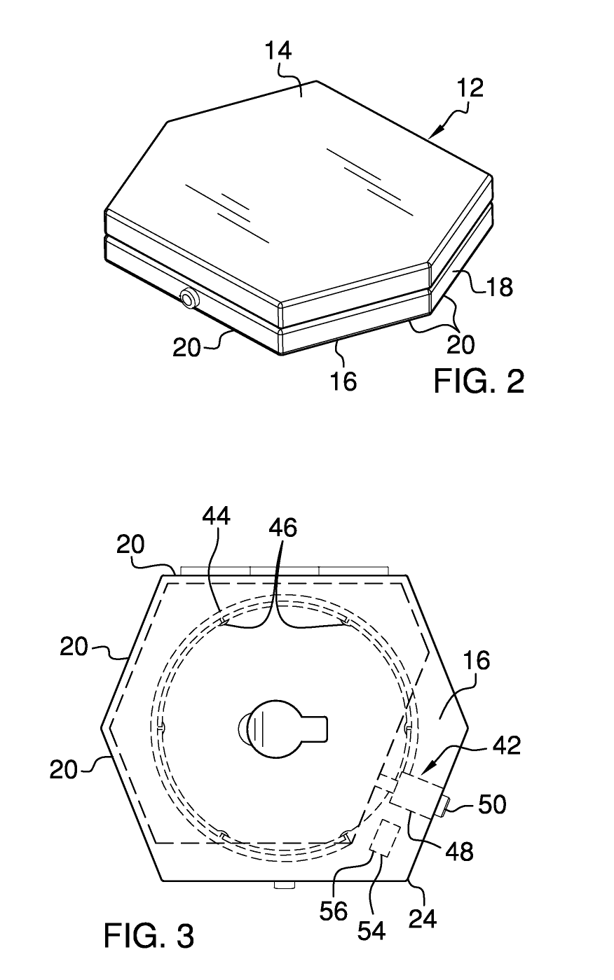

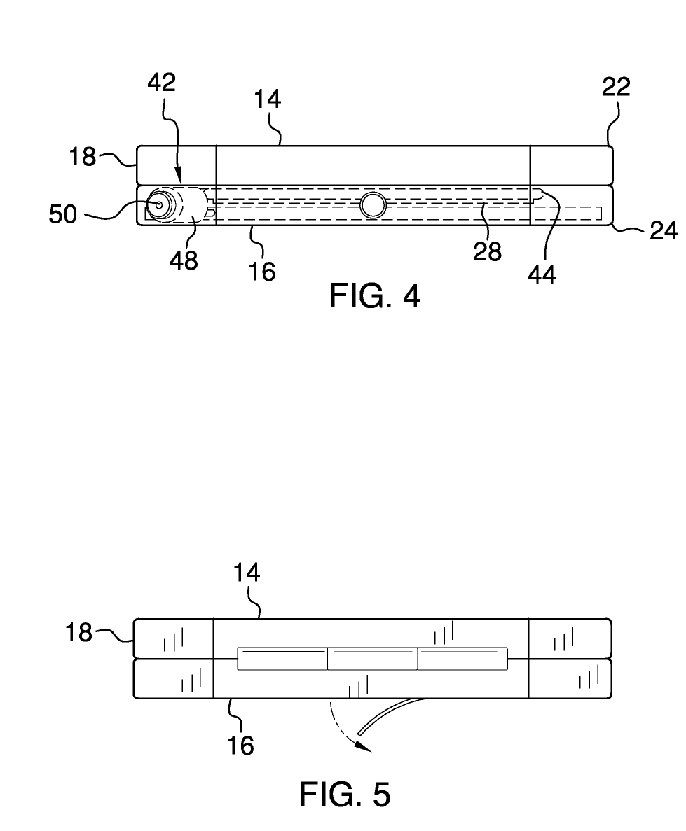

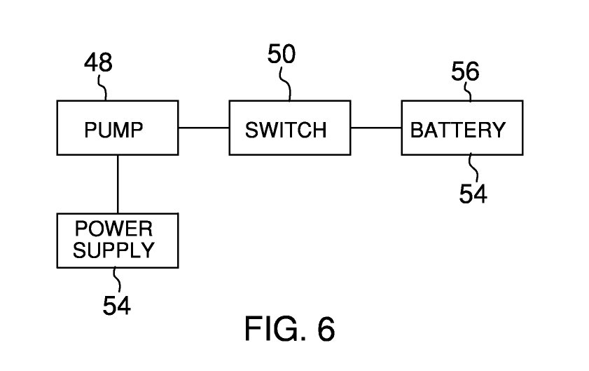

With reference now to the drawings, and in particular to FIGS. 1 through 6 thereof, a new container device embodying the principles and concepts of an embodiment of the disclosure and generally designated by the reference numeral 10 will be described.

As best illustrated in FIGS. 1 through 6, the contraception container assembly 10 generally comprises a housing 12 that has a top wall 14, a bottom wall 16 and a peripheral wall 18 extending between the top wall 14 and the bottom wall 16. The peripheral wall 18 has a plurality of intersecting sides 20 such that the housing 12 has a hexagonal shape. The peripheral wall 18 is divided between the top wall 14 and the bottom wall 16 to define a lid portion 22 of the housing 12 that is hingedly coupled to a container portion 24 of the housing 12. Each of the lid portion 22 and the container portion 24 has a respective mating surface 26.

The mating surface 26 of the container portion 24 has a first well 28 extending downwardly toward the bottom wall 16. The first well 28 may insertably receive a contraceptive device 30 and the contraceptive device 30 may comprise a NuvaRing manufactured by Merke & Co., Inc. The first well 28 has a lower bounding surface 32 and a perimeter bounding surface 34. The mating surface 26 of the lid portion 22 has a second well 31 extending upwardly toward the top wall 14. The housing 12 is positionable in a closed position having the mating surface 26 of the lid portion 22 abutting the mating surface 26 of the container portion 24 such that the first well 28 is aligned with the second well 31. The housing 12 is positionable in an open position to expose the first well 28.

A reservoir 34 is positioned within the housing 12 and the reservoir 34 is positioned within the container portion 24. The reservoir 34 is positioned between the lower bounding surface 32 of the first well 28 and the bottom wall 16 and the reservoir 34 may contain a fluid 36. The fluid 36 may be a saline solution or the like. The bottom wall 16 has an opening 38 extending into the reservoir 34 thereby facilitating the opening 38 to have the fluid 36 poured therein to fill the reservoir 34. A cap 40 is attached to the bottom wall 16 and the cap 40 selectively closes the opening 38.

A spraying unit 42 is attached to the housing 12 and the spraying unit 42 is in fluid communication between the reservoir 34 and the first well 28. The spraying unit 42 urges the fluid 36 from the reservoir 34 into the first well 28 thereby facilitating the contraceptive device 30 to be immersed in the fluid 36. The fluid 38 facilitates the contraceptive device 30 to be stored in a hygienic manner. The spraying unit 42 comprises a hose 44 that is positioned within the container portion 24. The hose 44 is positioned closer to the perimeter bounding surface 34 of the first well 28 than the peripheral wall 18 corresponding to the container portion 24. The hose 44 is substantially coextensive with the perimeter bounding surface 34.

A plurality of nozzles 46 is provided and each of the nozzles 46 is attached to the hose 44 and extends through the perimeter bounding surface 34. The nozzles 46 are spaced apart from each other and distributed along the hose 44 and each of the nozzles 46 is in fluid communication with the hose 44. A pump 48 is positioned within the container portion 24 and the pump 48 is positioned between the perimeter bounding surface 34 and the peripheral wall 18 corresponding to the container portion 24. The pump 48 is fluidly coupled between the reservoir 34 and the hose 44 such that the pump 48 urges the fluid 36 outwardly from the nozzles 46 into the first well 28. The pump 48 may be an electrically actuated fluid pump or the like.

A switch 50 is attached to the pump 48 and the switch 50 is electrically coupled to the pump 48. The switch 50 extends outwardly from the peripheral wall 18 corresponding to the container portion 24 and the switch 50 is engaged by a user to actuate the pump 48. A sensor 52 is attached to the mating surface 26 of the container portion 24 and the sensor 52 is electrically coupled to the pump 48. The sensor 52 is engaged by the mating surface 26 of the lid portion 22 to detect when the housing 12 is positioned in the closed position. The sensor 52 prevents the pump 48 from being actuated when the housing 12 is positioned in the open position. A power supply 54 is positioned within the container portion 24 and the power supply 54 is electrically coupled to the switch 50. The power supply 54 comprises at least one battery 56.

A lock 58 is attached to the housing 12 and the lock 58 is positioned on the mating surface 26 of the lid portion 22. The lock 58 engages the mating surface 26 of the container portion 24 when the housing 12 is positioned in the closed position thereby facilitating the housing 12 to be retained in the closed position. A release 60 is attached to the housing 12 and the release 60 is positioned on the peripheral wall 18 corresponding to the container portion 24. The release 60 may be engaged by the user to selectively disengage the lock 58 from the container portion 24 thereby facilitating the housing 12 to be positioned in the open position.

In use, the reservoir 34 is filled with the fluid 36 and the contraceptive device 30 is placed in the first well 28. The housing 12 is positioned in the closed position and the switch 50 is engaged to fill the first well 28 with the fluid 36. The contraceptive device 30 is stored within the housing 12 until such time that the contraceptive device 30 is utilized. The contraceptive device 30 is stored within the housing 12 whenever the contraceptive device 30 is not utilized in order to inhibit bacterial growth on the contraceptive device 30 and to prevent the contraceptive device 30 from contaminating extraneous objects.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A contraception container assembly for storing a contraceptive device in a hygienic manner, said assembly comprising:
a housing being configured to insertably receive a contraceptive device;
a reservoir being positioned within said housing, said reservoir being configured to contain a fluid; and
a spraying unit being attached to said housing, said spraying unit being in fluid communication with said reservoir wherein said spraying unit is configured to urge the fluid from said reservoir into said housing thereby facilitating the contraceptive device to be immersed in the fluid.

2. The assembly according to claim 1, wherein said housing has a top wall, a bottom wall and a peripheral wall extending between said top wall and said bottom wall, said peripheral wall having a plurality of intersecting sides wherein said housing has a hexagonal shape, said peripheral wall being divided between said top wall and said bottom wall to define a lid portion of said housing being hingedly coupled to a container portion of said housing.

3. The assembly according to claim 2, wherein each of said lid portion and said container portion has a respective mating surface, said mating surface of said container portion having a first well extending downwardly toward said bottom wall wherein said first well is, said first well having a lower bounding surface and a perimeter bounding surface, said mating surface of said lid portion having a second well extending upwardly toward said top wall.

4. The assembly according to claim 3, wherein said housing is positionable in a closed position having said mating surface of said lid portion abutting said mating surface of said container portion such that said first well is aligned with said second well, said housing being positionable in an open position to expose said first well.

5. The assembly according to claim 3, wherein said reservoir is positioned within said container portion, said reservoir being positioned between said lower bounding surface of said first well and said bottom wall, said bottom wall having an opening extending into said reservoir wherein said opening is configured to have the fluid poured therein to fill said reservoir.

6. The assembly according to claim 5, further comprising a cap being attached to said bottom wall, said cap selectively closing said opening.

7. The assembly according to claim 1, wherein:
said housing comprises a container portion and a peripheral wall, said housing having a first well, said first well having a perimeter bounding surface; and
said spraying unit comprises a hose being positioned within said container portion, said hose being positioned closer to said perimeter bounding surface of said first well than said peripheral wall corresponding to said container portion, said hose being substantially coextensive with said perimeter bounding surface.

8. The assembly according to claim 7, further comprising a plurality of nozzles, each of said nozzles being attached to said hose and extending through said perimeter bounding surface, said nozzles being spaced apart from each other and distributed along said hose, each of said nozzles being in fluid communication with said hose.

9. The assembly according to claim 8, further comprising a pump being positioned within said container portion, said pump being positioned between said perimeter bounding surface and said peripheral wall corresponding to said container portion, said pump being fluidly coupled between said reservoir and said hose such that said pump is configured to urge the fluid outwardly from said nozzles into said first well.

10. The assembly according to claim 9, further comprising a switch being attached to said pump, said switch being electrically coupled to said pump, said switch extending outwardly from said peripheral wall corresponding to said container portion wherein said switch is configured to be engaged by a user to actuate said pump.

11. The assembly according to claim 10, further comprising:
said container portion having a mating surface;
said housing having a lid portion, said lid portion having a mating surface;
said housing being positionable in a closed position and an open position;
a sensor being attached to said mating surface of said container portion, said sensor being electrically coupled to said pump, said sensor being engaged by said mating surface of said lid portion to detect when said housing is positioned in said closed position, said sensor preventing said pump from being actuated when said housing is positioned in said open position.

12. The assembly according to claim 1, further comprising:
said housing having a container portion;
a switch; and
a power supply being positioned within said container portion, said power supply being electrically coupled to said switch, said power supply comprising at least one battery.

13. The assembly according to claim 1, further comprising:
said housing having a lid portion and a container portion, each of said lid portion and said container portion having a respective mating surface, said housing being positioned in an open position and a closed position; and
a lock being attached to said housing, said lock is positioned on said mating surface of said lid portion, said lock engaging said mating surface of said container portion when said housing is positioned in said closed position thereby facilitating said housing to be retained in said closed position.

14. The assembly according to claim 13, further comprising:
said housing having a peripheral wall; and
a release being attached to said housing, said release selectively disengaging said lock, said release being positioned on said peripheral wall corresponding to said container portion wherein said release is configured to be engaged by a user.

15. A contraception container assembly for storing a contraceptive device in a hygienic manner, said assembly comprising:
a housing having a top wall, a bottom wall and a peripheral wall extending between said top wall and said bottom wall, said peripheral wall having a plurality of intersecting sides wherein said housing has a hexagonal shape, said peripheral wall being divided between said top wall and said bottom wall to define a lid portion of said housing being hingedly coupled to a container portion of said housing, each of said lid portion and said container portion having a respective mating surface, said mating surface of said container portion having a first well extending downwardly toward said bottom wall wherein said first well is configured to insertably receive a contraceptive device, said first well having a lower bounding surface and a perimeter bounding surface, said mating surface of said lid portion having a second well extending upwardly toward said top wall, said housing being positionable in a closed position having said mating surface of said lid portion abutting said mating surface of said container portion such that said first well is aligned with said second well, said housing being positionable in an open position to expose said first well;
a reservoir being positioned within said housing, said reservoir being positioned within said container portion, said reservoir being positioned between said lower bounding surface of said first well and said bottom wall, said reservoir being configured to contain a fluid, said bottom wall having an opening extending into said reservoir wherein said opening is configured to have the fluid poured therein to fill said reservoir;
a cap being attached to said bottom wall, said cap selectively closing said opening;
a spraying unit being attached to said housing, said spraying unit being in fluid communication between said reservoir and said first well wherein said spraying unit is configured to urge the fluid from said reservoir into said first well thereby facilitating the contraceptive device to be immersed in the fluid, said spraying unit comprising
a hose being positioned within said container portion, said hose being positioned closer to said perimeter bounding surface of said first well than said peripheral wall corresponding to said container portion, said hose being substantially coextensive with said perimeter bounding surface,
a plurality of nozzles, each of said nozzles being attached to said hose and extending through said perimeter bounding surface, said nozzles being spaced apart from each other and distributed along said hose, each of said nozzles being in fluid communication with said hose,
a pump being positioned within said container portion, said pump being positioned between said perimeter bounding surface and said peripheral wall corresponding to said container portion, said pump being fluidly coupled between said reservoir and said hose such that said pump is configured to urge the fluid outwardly from said nozzles into said first well,
a switch being attached to said pump, said switch being electrically coupled to said pump, said switch extending outwardly from said peripheral wall corresponding to said container portion wherein said switch is configured to be engaged by a user to actuate said pump,
a sensor being attached to said mating surface of said container portion, said sensor being electrically coupled to said pump, said sensor being engaged by said mating surface of said lid portion to detect when said housing is positioned in said closed position, said sensor preventing said pump from being actuated when said housing is positioned in said open position, and
a power supply being positioned within said container portion, said power supply being electrically coupled to said switch, said power supply comprising at least one battery;
a lock being attached to said housing, said lock being positioned on said mating surface of said lid portion, said lock engaging said mating surface of said container portion when said housing is positioned in said closed position thereby facilitating said housing to be retained in said closed position; and
a release being attached to said housing, said release being positioned on said peripheral wall corresponding to said container portion wherein said release is configured to be engaged by a user, said release selectively disengaging said lock from said container portion thereby facilitating said housing to be positioned in said open position.
